(19) United States
(12) Patent Application Publication
(10) Pub. No.: US 2026/0232997 A1
(43) Pub. Date: Aug. 13, 2026

(54) COCHLEAR IMPLANT WITH FULLY IMPLANTABLE MICROPHONE

(71) Applicant: Northwestern University, Evanston, IL (US)

(72) Inventors: Claus-Peter Richter, Skokie, IL (US); Joaquin Cury, Chicago, IL (US)

(21) Appl. No.: 19/156,300

(22) PCT Filed: Apr. 9, 2024
(86) PCT No.: PCT/US2024/023694

§ 371 Date: Aug. 13, 2025

Related U.S. Application Data
(60) Provisional application No. 63/495,165, filed on Apr. 10, 2023.

(51) Int. Cl. A61N 1/05; A61N 1/36; A61N 1/378

(52) U.S. Cl. CPC A61N 1/0541; A61N 1/36038; A61N 1/3785

(57) ABSTRACT

A cochlear implant has a microphone unit having a housing and a bone anchor attached thereto. The bone anchor is configured to secure the microphone unit to bone that surrounds a tympanic cavity of a user such that the microphone unit is positioned within the tympanic cavity. The implant includes a primary microphone mounted within the housing to receive acoustic energy from an external source. The implant also includes a processor in communication with the microphone unit. The processor is configured to process the acoustic energy and to generate an electrical signal corresponding to the acoustic energy. The implant also includes a plurality of electrodes mounted on a cochlea of the user and in communication with the processor. The processor activates one or more of the plurality of electrodes to correspond to the generated electrical signal to cause a hearing sensation for the user that corresponds to the acoustic energy.

### CROSS-REFERENCE TO RELATED APPLICATION

The present application claims the priority benefit of U.S. Provisional Patent App. No. 63/495,165 filed on Apr. 10, 2023, the entire disclosure of which is incorporated herein by reference.

### STATEMENT REGARDING FEDERALLY SPONSORED RESEARCH

This invention was made with government support under R01 DC18666 awarded by the National Institutes of Health. The government has certain rights in the invention.

### BACKGROUND

A cochlear implant refers to a device that enables individuals with severe hearing loss to perceive sounds. The cochlear implant is a surgically implanted neuroprosthesis that is used to stimulate a patient's auditory nerve. The stimulation is generally performed by way of electrodes that are placed in the cochlea of the inner ear. Specifically, the electrodes are activated in response to audio signals that are received by a microphone attached to the outer ear of the patient. The received audio signals are provided to an implanted receiver, which in turn activates the electrodes to stimulate the auditory nerve. Stimulation of the auditory nerve directs signals to the brain, which the brain interprets as sound.

### SUMMARY

An illustrative cochlear implant has a microphone unit having a housing and a bone anchor attached thereto. The bone anchor is configured to secure the microphone unit to bone that surrounds a tympanic cavity of a user such that the microphone unit is positioned within the tympanic cavity. The implant includes a primary microphone mounted within the housing to receive acoustic energy from an external source. The implant also includes a processor in communication with the microphone unit. The processor is configured to process the acoustic energy and to generate an electrical signal corresponding to the acoustic energy. The implant also includes a plurality of electrodes mounted on a cochlea of the user and in communication with the processor. The processor activates one or more of the plurality of electrodes to correspond to the generated electrical signal to cause a hearing sensation for the user that corresponds to the acoustic energy.

In one embodiment, the housing comprises a titanium housing that encases at least a portion of the primary microphone. In another embodiment, the titanium housing includes an opening that is covered with a membrane that allows pressure waves to pass through to the primary microphone. In one embodiment, the microphone has an input port, the input port is oriented to face the membrane, and the membrane is oriented to face a tympanic membrane of the user.

In another embodiment, the acoustic energy received by the primary microphone is first acoustic energy, and the microphone unit also includes a secondary microphone that is configured to receive second acoustic energy. The second acoustic energy reaches the tympanic cavity through the bone of the user. In an illustrative embodiment, an input port of the secondary microphone is oriented to face the bone that surrounds the tympanic cavity. In another embodiment, the processor is configured to process the first acoustic energy and the second acoustic energy to generate the electrical signal, where the electrical signal corresponds to the first acoustic energy.

In another embodiment, an accelerometer is mounted on or adjacent to the microphone unit, and the accelerometer is configured to receive third acoustic energy that is transferred through the bone. In such an embodiment, the processor is configured to process the first acoustic energy, the second acoustic energy, and the third acoustic energy to generate the electrical signal, where the electrical signal corresponds to the first acoustic energy. In one embodiment, the accelerometer is mounted within the bone anchor that secures the microphone unit to the bone. In another embodiment, the processor applies a low frequency filter to filter out the acoustic energy that is lower than 100 Hertz. In another embodiment, the implant includes one or more implanted energy harvesters that provide power to the microphone unit responsive to one or more of muscle contractions of the user, blood steam pulsations of the user, and temperature gradients of the user. In one embodiment, damping material mounted to an exterior surface of the housing, and the bone anchor is attached to the damping material.

An illustrative method of forming a cochlear implant includes forming a housing for a microphone unit, where the housing is sized to fit within a tympanic cavity of a user. The method includes attaching a bone anchor to the housing, where the bone anchor is sized to secure the housing to bone that surrounds the tympanic cavity of the user. The method also includes mounting a primary microphone within the housing to receive acoustic energy that is received from a source external to the user. The method also includes placing a processor into communication with the microphone unit, where the processor is configured to process the acoustic energy and to generate an electrical signal corresponding to the acoustic energy. The method further includes placing a plurality of electrodes into communication with the processor, where the plurality of electrodes are configured to deliver the electrical signal.

In one embodiment, the method includes mounting a secondary microphone within the housing, where the acoustic energy received by the primary microphone comprises first acoustic energy, and where the secondary microphone is positioned to receive second acoustic energy from the bone that surrounds the tympanic cavity. The method can also include mounting an accelerometer in or adjacent to the housing, where the accelerometer is positioned to receive third acoustic energy received from the bone that surrounds the tympanic cavity. In one embodiment, the accelerometer is mounted within the bone anchor. In another embodiment, the processor is configured to process the first acoustic energy, the second acoustic energy, and the third acoustic energy to generate the electrical signal such that the electrical signal corresponds to the first acoustic energy. In another embodiment, the method includes attaching damping material to an exterior surface of the housing, where the bone anchor is attached to the damping material.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A is an overview of a cochlear implant system in accordance with an illustrative embodiment.

FIG. 1B depicts an enlarged, cross-sectional view of the cochlear implant system in accordance with an illustrative embodiment.

FIG. 2A is a cross-sectional view of an ear, along with a breakout view of the middle ear in accordance with an illustrative embodiment.

FIG. 2B is an enlarged view of the middle ear in accordance with an illustrative embodiment.

FIG. 3 is a diagram that depicts the acoustic energies that may be experienced by a cochlear implant in accordance with an illustrative embodiment.

FIG. 4 depicts a cochlear implant system that includes a computing device in accordance with an illustrative embodiment.

### DETAILED DESCRIPTION

A cochlear implant (CI) is a small, complex electronic device that can be used to restore some hearing in individuals that are severely-to-profoundly deaf. More specifically, a cochlear implant is a device that records the acoustic signals present in the environment, processes this information, and encodes it into sequences of electrical pulses that are delivered to various places along the cochlea of the patient. In traditional devices, sound capture and processing of the information is done in a component of the CI which is placed externally behind the ear. Such external components can cause comfort issues, and some users do not like the aesthetic appearance of these components.

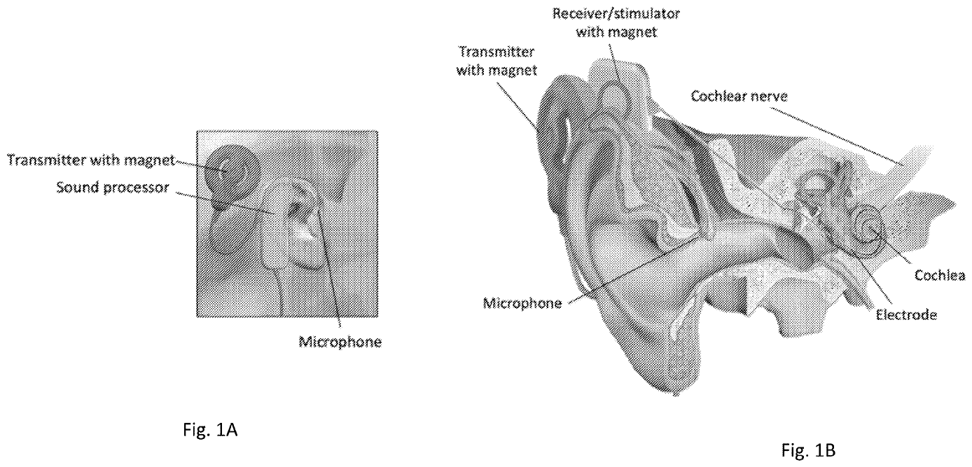

Traditional CI systems and their limitations are described below, along with the newly proposed system that overcomes the limitations of traditional implants. FIG. 1A is an overview of a cochlear implant system in accordance with an illustrative embodiment. FIG. 1B depicts an enlarged, cross-sectional view of the cochlear implant system in accordance with an illustrative embodiment. As shown, the cochlear implant includes external components and internal components. The external components include an externally worn microphone, speech processor, and transmission coil. The internal components include an implanted receiver coil, stimulator unit, and cochlear implant electrode array. As also shown, two magnets are used to align the transmission coils of the system.

The microphone transforms received sound into an electrical signal. The generated electrical signal is transmitted as an input to the speech processor, which converts the analog voltage from the microphone to a digital signal, separates the complex acoustical signal into frequency bands, and generates a sequence of electrical pulses and corresponding amplitudes. The electrical pulses and corresponding amplitudes are delivered via the cochlear implant array of electrodes to the cochlea to evoke action potentials (APs) of the auditory nerve. In one embodiment, the pulse information (timing and amplitude) is transmitted via an RF connection to the implanted receiver. The implanted receiver (or stimulator) has multiple current sources that produce charge-balanced electrical pulses delivered to the cochlea according to the times and amplitudes transmitted from the speech processor.

While CIs are among the most successful neural prostheses, with approximately 500,000 implanted users worldwide, traditional cochlear implants have several challenges. The performance of individual users varies largely, and noisy listening environments, music, and tonal languages can be a challenge to all individuals with CIs. One reason for the varied results is that the electrical current spreads during electrical stimulation, resulting in overlaps. These overlaps lead to the simultaneous stimulation of multiple neuron populations, which results in distortion of the transmitted information. Therefore, new neural stimulation modalities, novel cochlear implant electrode designs, and coding strategies are being explored to increase the number of independent frequency bands onto which acoustic information is encoded. Another challenge with traditional CIs in which the processors are not fully implanted relates to possible trauma and the effects of head movement and gravity. The latter limits the a user's daily activities. Furthermore, an external sound processor cannot be worn in bed, which has clear safety implications and can further heighten self-awareness of hearing loss. In addition to performance, the consciousness of wearing a CI and its visibility frequently impact the daily life of patients.

The established CI companies have attempted to develop fully implantable cochlear implants (fiCI) to overcome some of the above-described limitations of traditional CIs. In one implementation, a sound processor, microphone, and battery are implanted. As an added benefit in such a system, no magnet is required as in conventional CIs. A challenge of such a fully implantable system is the microphone and its placement because the microphone records the sound transmitted to the ear, but also receives the sound of the heartbeat, breathing sounds, and the person's voice. As such, the technology used by the microphone and its mounting location are very important aspects.

With respect to microphone mounting location, various different locations for an implantable microphone have been explored. These locations can be grouped into four classifications (subcutaneous microphones, within the CI, middle ear microphones, and inner ear microphones). To implement subcutaneous microphones, either the microphone is just covered directly by the skin, or the microphone is attached to the bone and covered directly by the skin. Within the CI refers to a microphone that is mounted at some location within the cochlear implant itself. A microphone placed in the middle ear can be placed so that it touches the middle ear ossicles to detect the sound-induced vibrations, and can use optical techniques to measure sound-induced vibrations of the ossicles. Such a microphone is positioned behind the tympanic membrane. An inner ear microphone utilizes mechano-sensors placed into the scala tympani to measure pressure.

Various different types of microphones have also been considered for use in cochlear implants. Condenser microphones include a diaphragm that, when deformed by sound pressure, generates an electrical signal from a charge of a capacitor that is formed by the microphone's membrane and its corresponding backplate. The most commonly encountered capacitive microphone is the electret condenser microphone (ECM), which uses a material that is permanently polarized called electret, which is a dielectric.

An electromagnetic microphone is one of the first sensors implanted in an ex-vivo middle ear. The sensor of an electromagnetic microphone includes a small titanium-encapsulated neodymium iron-boron magnet glued to the head of the malleus (an ossicle). The magnet interacts with an electric coil placed on a titanium shaft that is supported in the temporal bone at a distance of 0.5-1 mm from the magnet. Electromagnetic microphones convert the velocities of the ossicles into an electrical signal. These sensors tend to have a smoother frequency response and respond better at low frequencies than piezoelectric microphones. However, there are also limitations of electromagnetic microphones. Specifically, placement in the middle ear is difficult, and the power consumption is up to two orders of magnitude larger than other microphones.

An optical microphone measures the vibration of the tympanic membrane (or one of the ossicles) through the reflection of a laser beam radiated by an optical fiber. The incident and reflected beams are captured by two photodiodes, transforming them into an electric signal. The main limitations of this type of microphone are its low sensitivity and the amplitude of the small vibrations of structures in the middle ear.

Another type of microphone that can potentially be used in a CI is a piezoresistive microphone. Piezoresistive materials exhibit a change of resistivity when a mechanical strain is applied to them. The first microphone of this type was implanted in the incus (an ossicle) in the middle ear. This microphone was based on a piezoresistive accelerometer. It was fabricated with silicon proof mass suspended by a thin, flexible beam. Piezo-resistors were coupled on each lateral face of the beam so that the acceleration of the incus induces differential strain by the shear stress induced on the elements. This type of microphone was tested in models and human cadaveric temporal bones, but as with the other above-discussed microphones, no studies have been reported on patients.

A piezoelectric microphone uses piezoelectric materials, which are special crystals that generate an electrical voltage when deformed. The most suitable piezoelectric transducers are built as a ‘bimorph,’ in which two crystals are sandwiched together, effectively multiplying the deformation. Larger output occurs when the transducer is formed in the shape of a cantilever with one end fixed and the other mobile. At present, the only commercialized middle ear microphone (piezoelectric) is part of the CI from Envoy Medical (USA). The sensor has to be coupled with the body of the incus (one of the ossicles), transducing movements of the disarticulated ossicular chain into electrical signals. While this sensor has low power consumption and a small size, it exhibits some limitations. For instance, too much or too little force between the piezoelectric microphone and the ossicle will result in a conductive loss and therefore reduced sensitivity. Furthermore, there is a trade-off between sensitivity and the resonance frequency. Specifically, the shorter the device, the higher the resonant frequency is for an equivalent mass. Conversely, the longer the device is, the more force it generates. Piezoelectric microphones must be built to precise dimensions to optimize their resonance frequency and sensitivity. At present, it is not possible to have a piezoelectric microphone with sufficient sensitivity and a resonant frequency below the range of normal hearing.

In the past, microphones placed under the skin and fixed to the bone perform poorly compared to an external microphone because of inadvertent inputs to the microphone. Body-produced sounds, such as scratching the scalp, breathing, heartbeats, and the user's voice interfere with the sound to be recorded by the microphone and limit performance of the system. The microphone sensitivity is directly proportional to the soft tissue thickness overlying it. The skin acts as a low-pass filter. As a result, the overlying skin/tissue drastically reduces acoustic information in the hearing range as well as the dynamic range of the microphone, producing sound distortion and signal attenuation. Moreover, sound localization in subcutaneous microphones is limited. Microphones inside the CI have a lower performance quality than those under the skin because, in this case, they have an additional layer (casing) that affects how sound is perceived.

As noted above, middle-ear microphones convert the movement of the tympanic membrane or ossicles into an electrical signal. Their performance is better than microphones under the skin. However, middle-ear microphones exhibit some constraints, such as small input signals, high noise, and limited bandwidth, consequently reducing the microphone's dynamic range and sensitivity. Intracochlear microphones (IMs) have recently emerged as a potential alternative to subcutaneous and middle-ear microphones. Even though IMs are devices under ongoing research, a major challenge is that inserting an additional component (besides the cochlear electrode) within the confined intracochlear space remains problematic.

In an effort to overcome the issues found in traditional systems, the inventors have explored a fully implantable CI that does not include any visible external components. For a fully implantable device, the microphone and the speech processor are implanted as well as the other components of the system (i.e., electrodes, stimulator, etc.). One problem with such a configuration is that the acoustical signal transmitted to the ear is largely influenced by tissue above the microphone. To address this challenge, one approach is to capture the signal from the middle ear ossicles or the tympanic membrane by mechanical and optical approaches. However, the small vibrations of the middle ear ossicles limit the dynamic range for which sound levels can be recorded and limit the signal-to-noise ratio, not overcoming the microphone issue. Described herein is a new approach, in which a microphone unit is placed in the air filled space behind the middle ear, avoiding the effect of tissue in front of the microphone.

More specifically, described herein is an improved cochlear implant with a fully implantable microphone assembly that overcomes the limitations of traditional cochlear implant systems. Important parameters for the microphone of the proposed system include sensitivity, dynamic range, and size. In one embodiment, the system uses compact micro-electro-mechanical systems (MEMS) microphones, which have a reliable monolithic structure and significantly lower mechanical vibration, power consumption, and noise interference compared to other types of microphones. MEMS microphones also offer a better signal-to-noise ratio (SNR) and support a wider operating temperature range than other traditional microphones.

In an illustrative embodiment, one or more MEMS microphones are encapsulated in a housing that can be made of titanium or any other biocompatible material(s). The housing is close to the microphone(s) to keep the footprint small. The housing can include an input port for each of the microphones. Additionally, one side of the housing, opposite the input port of a first (or primary) microphone, can include a very thin membrane to allow the pressure waves to be transmitted to the first microphone. While the input port of the first microphone is located close to and pointing towards the tympanic membrane, the remainder of the housing is covered with damping materials having high-pass characteristics.

In an illustrative embodiment, the damping materials covering the microphone housing also incorporate an accelerometer, which directly interacts with the bone. Any type of accelerometer known in the art may be used. The accelerometer is used to record the sound transmitted through the bone, and a second microphone is mounted in the housing with its input port pointing toward the bone to reduce inadvertent noise further. In one embodiment, noise reduction can be achieved by the hardware configuration, where the noise signal from the accelerometer and the microphones are combined by a processor to cancel the noise signal. Alternatively, the cancellation can be achieved through software using the input from the two microphones and the accelerometer. Thus, the 2 microphones mounted in the housing are essentially combined to form an active noise-canceling system.

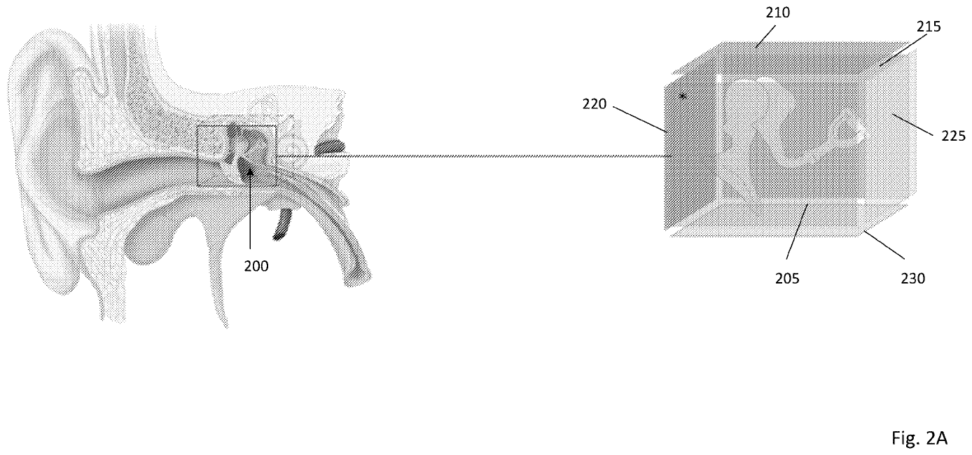

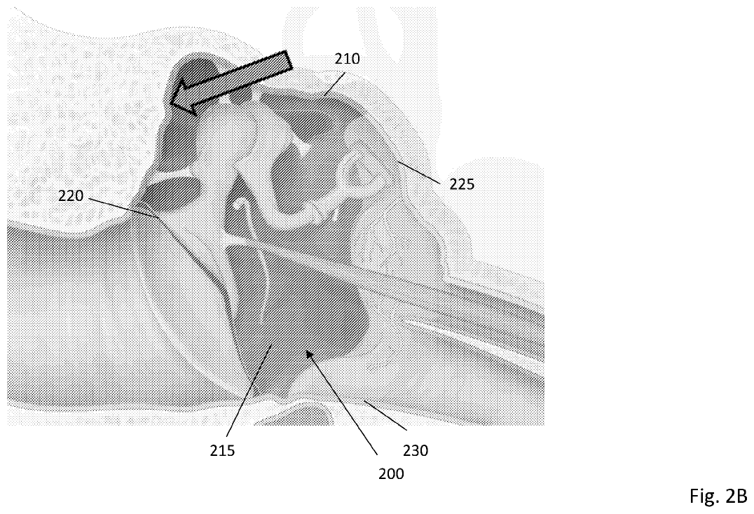

In another illustrative embodiment, the microphone housing can be placed in the middle ear, particularly next to the mastoid cavity (close to the attachment of the posterior incudal ligament). The middle ear is a closed cavity, and sound can therefore be perceived in all directions. FIG. 2A is a cross-sectional view of an ear, along with a breakout view of the middle ear in accordance with an illustrative embodiment. FIG. 2B is an enlarged view of the middle ear in accordance with an illustrative embodiment. Shown in the figures are features of the tympanic cavity 200 that forms the middle ear. Specifically, the figures show an anterior wall 205 of the tympanic cavity (visible only in FIG. 2A), a roof (or cranial border) 210 of the tympanic cavity, a posterior wall (or dorsal border) 215 of the tympanic cavity, a lateral wall (or tympanon or lateral border) 220 of the tympanic cavity, a medial wall (or medial border) 225 of the tympanic cavity, and a floor (or caudal border) 230 of the tympanic cavity. The arrow in FIG. 2B shows the mounting location of the microphone(s) and accelerometer. In an illustrative embodiment, the microphone housing includes a bone anchor that mounts the housing to bone. The accelerometer can be incorporated within the bone anchor in one embodiment. Alternatively, the accelerometer can otherwise be mounted on the housing of the microphone assembly such that the accelerometer receives acoustic energy through the bone.

Contrary to other proposed middle-ear sensors (described above) in which the microphone is in contact with structures such as the ossicles or the tympanic membrane, the proposed microphone housing is fixed in the bone to take advantage of the sound resonance in the middle-ear cavity. The placement of the primary microphone overcomes several issues with contemporary systems, including the limited frequency range of the acoustic signal caused by the low pass filter of the skin, the reduced dynamic range of the microphone originating from the damping of the acoustic signal by the skin, and the low contact area between the microphone and the ossicles and tympanic membrane, which can affect its sensitivity.

As discussed, using the proposed microphone placement, the noise will be canceled by either hardware configuration and/or software, depending on the implementation. With respect to hardware configuration, a first primary microphone records the energy of the sound reaching the middle ear cavity through the tympanic membrane. The accelerometer fixed in the bone measures the body noise transmitted across the bone. The secondary microphone facing the bone captures the body noise that reaches the middle ear cavity. With respect to software, in one embodiment an algorithm filtering low-frequency bands under 100 Hz (related to the body noise) is used to further improve noise rejection and post-processing. Alternatively, a different low-frequency filter threshold may be used, such as 80 Hz, 90 Hz, 120 Hz, etc.

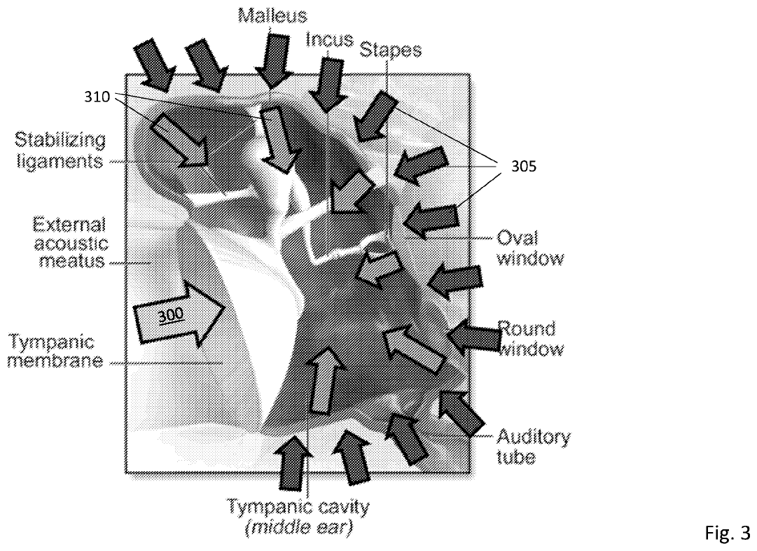

FIG. 3 is a diagram that depicts the acoustic energies that may be experienced by a cochlear implant in accordance with an illustrative embodiment. The acoustic energies are depicted in the form of arrows, and an arrow 300 corresponds to the acoustic energy E₁ transmitted through the tympanic membrane to the middle ear cavity. The outermost arrows 305 represent the acoustic energy E₂ corresponding to body noise (e.g., head movement, breathing, heart beating, etc.) transmitted through the bone. The inner arrows 310 describe the acoustic energy E₃ corresponding to the body noise that passed through the bone and reached the middle ear cavity. The sum of these three energies determines the total energy (Eₜ) that the implantable microphone system will receive, as indicated by Equation 1 below. The first microphone facing the tympanic membrane will pick up E₁, the accelerometer fixed in the bone will pick up E₂, and the second microphone facing the bone will pick up E₃.

$${E}_{t} = {E}_{1} + {E}_{2} + {E}_{3} \qquad Equation 1$$

Various embodiments to provide the power for the microphones and accelerometer are also envisioned. One option is to utilize a rechargeable battery for the microphone unit alone that can be implanted in a small drill-out directly with the microphone housing. A second option is to have the power directly supplied by the CI. Another option is to use implantable energy harvesters, which are devices that generate electrical signals that can be stored and used to power an implantable device. Several external sources can be used, such as electromagnetic radiation and ultrasound. However, the system can also take advantage of different strategies. For instance, it is possible to generate a sufficient voltage difference to maintain the microphone's functioning by using muscle contraction (respiration, head movement, etc.), blood steam pulsations, and temperature gradients within the body.

The connection between the microphone and the implanted CI can be established as a wired connection or as a wireless connection. Wireless technologies are still more commonly used for research, with limited applications in clinical implantable devices such as CIs. In this regard, a broad range of wireless technologies and communication protocols could fit the vast array of medical applications, such as WIFI, ZigBee, Thread, Bluetooth, etc. In embodiments that use a wireless connection, it is crucial to employ connectivity that has a low power consumption, straightforward connectivity with devices (i.e., the CI, mobile phones, and computers), and more importantly, safe and secure communication. In this regard, bidirectional communication between the CI and the microphone unit is preferred if security is a priority for the application. Nevertheless, special attention must be considered for scenarios where an authorized person or device wants to connect the microphone or implant and make changes, altering the functionality. With these considerations, Bluetooth Low Energy (BLE) is a suitable option for an implantable microphone.

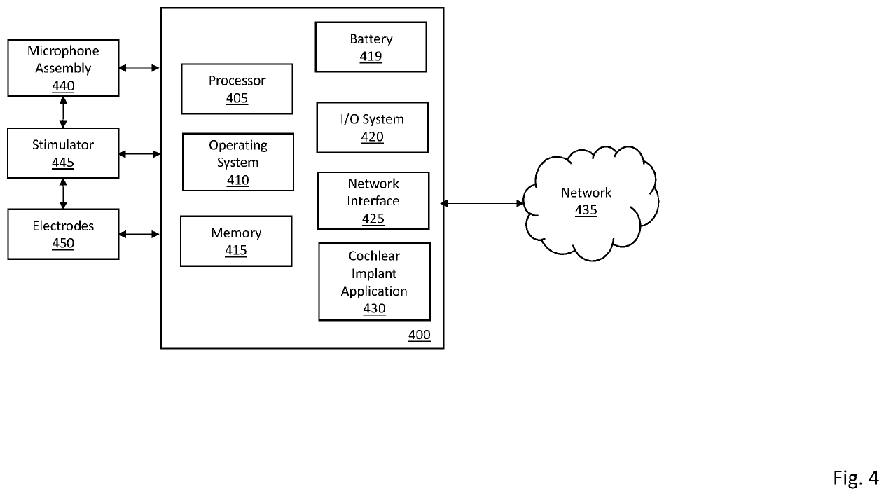

In an illustrative embodiment, any of the operations or calculations described herein can be performed by a computing system that includes a processor, a memory, a transceiver (receiver and/or transmitter), etc. The operations can be stored as computer-readable instructions in the memory. Upon execution by the processor of the computer-readable instructions, the computing system performs the operations, calculations, etc. described herein. As an example, FIG. 4 depicts a cochlear implant system that includes a computing device 400 in accordance with an illustrative embodiment. In one embodiment, the computing device 400 can be in wired communication with all of the other components of the system. Alternatively, one or more portions of the computing device 400 can be in wireless communication with other system components via a network 435 and/or through a direct wireless connection.

In addition to the computing device 400, the cochlear implant system also includes a microphone assembly 440, a stimulator 445, and electrodes 450. The microphone assembly 440 can include a housing that contains a first (primary) microphone and a second (secondary) microphone). The housing can include an input port for each of the primary and secondary microphones, and in one embodiment a membrane can be used to cover the input ports. Damping material can be mounted/applied to the housing to improve acoustics of the microphones. Additionally, the microphone assembly 440 can include an accelerometer incorporated therein. In one embodiment, the accelerometer is embedded in the damping material. The microphone assembly 440 mounts directly to bone such that the accelerometer is in contact with the bone and able to receive signals through the bone.

In an illustrative embodiment, the microphone assembly 440 is positioned within the ear so that it is not externally visible on the user. The microphone assembly 440 receives audio energy (i.e., sound, vibrations, etc.) through the primary microphone, the secondary microphone, and the accelerometer. As discussed herein, these audio energy signals can be processed to remove signal noise, such as internal sounds that result from heartbeats, breathing, etc. The processed audio signals are converted into electrical impulses via the stimulator 445, a processor 405, and/or a cochlear implant application 430 of the system. The stimulator 445 generates the electrical pulses, which are provided to the electrodes 450. In an illustrative embodiment, the electrodes 450 are in the form of an electrode array that is spread out along the auditory nerve of the user. The electrodes 450 deliver the electrical signals to selected portions of the auditory nerve to evoke action potentials (APs) of the auditory nerve, which the user experiences as sound.

The computing device 400 includes the processor 405, an operating system 410, a memory 415, a battery 419, an input/output (I/O) device 420, a network interface 425, and the cochlear implant application 430. In alternative embodiments, the computing device 400 may include fewer, additional, and/or different components. The components of the computing device 400 communicate with one another via one or more buses or any other interconnect device.

The processor 405 of the computing device 400 can be in electrical communication with and used to control any of the device components described herein, such as the microphone assembly 440, the stimulator 445, the electrodes 450, etc. The processor 405 can be any type of computer processor known in the art, and can include a plurality of processors and/or a plurality of processing cores. The processor 405 can include a controller, a microcontroller, an audio processor, a hardware accelerator, a digital signal processor, etc. Additionally, the processor 405 may be implemented as a complex instruction set computer processor, a reduced instruction set computer processor, an x86 instruction set computer processor, etc. The processor 405 is used to run the operating system 410, which can be a custom operating system specific to the requirements of the proposed device.

The operating system 410 is stored in the memory 415, which is also used to store programs, device data, user information, algorithms, network and communications data, peripheral component data, and other operating instructions. The memory 415 can be one or more memory devices that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. The battery 419 is used to power components of the cochlear implant system. Any battery of appropriate size and capacity may be used. In an alternative embodiment, a power source other than a battery may be used.

The I/O system 420, or user interface, is the framework which enables users (and peripheral devices) to interact with the computing device 400. The I/O system 420 can include one or more buttons, a microphone, software, etc. that allow the user to interact with and control the computing device 400. The I/O system 420 can be used to upgrade system software in one embodiment. The I/O system 420 also includes circuitry and a bus structure to interface with peripheral computing components such as the battery 419 or other power sources, etc.

The network interface 425 includes transceiver circuitry that allows the computing device 400 to transmit and receive data to/from other devices such as user device(s), remote computing devices, servers, websites, etc. The network interface 425 enables communication through the network 435, which can be one or more communication networks. The network 435 can include a cable network, a fiber network, a cellular network, a wi-fi network, a landline telephone network, a microwave network, a satellite network, etc. The network interface 425 also includes circuitry to allow device-to-device communication such as near field communication (NFC), Bluetooth® communication, etc.

The cochlear implant application 430 can include software and algorithms (e.g., in the form of computer-readable instructions) which, upon activation or execution by the processor 405, performs any of the various operations described herein such as controlling the microphone assembly 440, controlling the stimulator 445, controlling the electrodes 450, receiving sensed data, performing analyses of sensed data, removing noise from the sound data, determining appropriate electrical signals to generate corresponding to sound received by the microphone assembly, generating electrical signals corresponding to received sound, etc. The cochlear implant application 430 can utilize the processor 405 and/or the memory 415 as discussed above.

In summary, described herein is a cochlear implant that includes a small fully implantable, sensitive microphone assembly. In one embodiment, the microphone assembly is encapsulated in a thin platinum housing that is placed in the open cavity of the middle ear. The microphone unit includes a bone anchor, and the bone anchor portion of the microphone unit includes an accelerator which records the bone-conducted background sound/noise from the human body. In particular, the patient's own voice and body sound are captured to be removed either through a hardware configuration or by software correction from the incoming sound signal. A dedicated secondary microphone in the microphone assembly is utilized to receive any sounds which pass through the bone to complement use of the accelerometer. A primary microphone in the microphone assembly is configured and positioned to receive (normal) sounds through the ear canal, such as music, speech, background noise, etc. The design of the microphone assembly and the placement of the microphones therein will overcome several challenges with contemporary art, including the limited frequency range of the acoustic signal caused by the low pass filter of the skin, the reduced dynamic range of the microphone originating from the damping of the acoustic signal by the skin, the reduction of the body noise by adding a special attachment of the microphone to the bone with high pass filter function, and the possibility for hardware and/or software-based noise cancellation.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A cochlear implant comprising:
a microphone unit that includes:
a housing;
a bone anchor attached to the housing and configured to secure the microphone unit to bone that surrounds a tympanic cavity of a user such that the microphone unit is positioned within the tympanic cavity; and
a primary microphone mounted within the housing to receive acoustic energy that is received from a source external to the user;
a processor in communication with the microphone unit, wherein the processor is configured to process the acoustic energy and to generate an electrical signal corresponding to the acoustic energy; and
a plurality of electrodes mounted on a cochlea of the user and in communication with the processor, wherein the processor activates one or more of the plurality of electrodes to correspond to the generated electrical signal to cause a hearing sensation for the user that corresponds to the acoustic energy.

2. The cochlear implant of claim 1, wherein the housing comprises a titanium housing that encases at least a portion of the primary microphone.

3. The cochlear implant of claim 2, wherein the titanium housing includes an opening that is covered with a membrane that allows pressure waves to pass through to the primary microphone.

4. The cochlear implant of claim 3, wherein the microphone has an input port, and wherein the input port is oriented to face the membrane, and wherein the membrane is oriented to face a tympanic membrane of the user.

5. The cochlear implant of claim 1, wherein the acoustic energy received by the primary microphone is first acoustic energy, and wherein the microphone unit also includes a secondary microphone that is configured to receive second acoustic energy.

6. The cochlear implant of claim 5, wherein the second acoustic energy reaches the tympanic cavity through the bone of the user.

7. The cochlear implant of claim 5, wherein an input port of the secondary microphone is oriented to face the bone that surrounds the tympanic cavity.

8. The cochlear implant of claim 5, wherein the processor is configured to process the first acoustic energy and the second acoustic energy to generate the electrical signal, wherein the electrical signal corresponds to the first acoustic energy.

9. The cochlear implant of claim 5, further comprising an accelerometer mounted on or adjacent to the microphone unit, wherein the accelerometer is configured to receive third acoustic energy that is transferred through the bone.

10. The cochlear implant of claim 9, wherein the processor is configured to process the first acoustic energy, the second acoustic energy, and the third acoustic energy to generate the electrical signal, wherein the electrical signal corresponds to the first acoustic energy.

11. The cochlear implant of claim 9, wherein the accelerometer is mounted within the bone anchor that secures the microphone unit to the bone.

12. The cochlear implant of claim 1, wherein the processor applies a low frequency filter to filter out the acoustic energy that is lower than 100 Hertz.

13. The cochlear implant of claim 1, further comprising one or more implanted energy harvesters that provide power to the microphone unit responsive to one or more of muscle contractions of the user, blood steam pulsations of the user, and temperature gradients of the user.

14. The cochlear implant of claim 1, further comprising damping material mounted to an exterior surface of the housing, wherein the bone anchor is attached to the damping material.

15. A method of forming a cochlear implant, the method comprising:
forming a housing for a microphone unit, wherein the housing is sized to fit within a tympanic cavity of a user;
attaching a bone anchor to the housing, wherein the bone anchor is sized to secure the housing to bone that surrounds the tympanic cavity of the user; and
mounting a primary microphone within the housing to receive acoustic energy that is received from a source external to the user;
placing a processor into communication with the microphone unit,
wherein the processor is configured to process the acoustic energy and to generate an electrical signal corresponding to the acoustic energy; and
placing a plurality of electrodes into communication with the processor, wherein the plurality of electrodes are configured to deliver the electrical signal.

16. The method of claim 15, further comprising mounting a secondary microphone within the housing, wherein the acoustic energy received by the primary microphone comprises first acoustic energy, and wherein the secondary microphone is positioned to receive second acoustic energy from the bone that surrounds the tympanic cavity.

17. The method of claim 16, further comprising mounting an accelerometer in or adjacent to the housing, wherein the accelerometer is positioned to receive third acoustic energy received from the bone that surrounds the tympanic cavity.

18. The method of claim 17, wherein the accelerometer is mounted within the bone anchor.

19. The method of claim 17, wherein the processor is configured to process the first acoustic energy, the second acoustic energy, and the third acoustic energy to generate the electrical signal, wherein the electrical signal corresponds to the first acoustic energy.

20. The method of claim 15, further comprising attaching damping material to an exterior surface of the housing, wherein the bone anchor is attached to the damping material.
